(19) United States
(12) Patent Application Publication
(10) Pub. No.: US 2008/0119719 A1
(43) Pub. Date: May 22, 2008

(54) TEMPLATES FOR ASSESSING BONE QUALITY AND METHODS OF USE THEREOF

(75) Inventors: Maria-Grazia Ascenzi, Santa Monica, CA (US); Angela Favia, Bari (IT)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/842,784

(22) Filed: Aug. 21, 2007

Related U.S. Application Data
(60) Provisional application No. 60/839,312, filed on Aug. 21, 2006.

(51) Int. Cl. C12Q 1/00; A61B 5/05

(52) U.S. Cl. 600/410; 435/4

(57) ABSTRACT

The present invention relates to the preparation and use of novel bone templates that can be prepared using a comprehensive approach to observing microstructural features of bone, including trabecular thickness and trabecular density. These features are assessed in regions of interest in a bone (e.g., proximal femur, distal femur, wrist, spine, etc.) as observed using digital radiographic techniques or clinical imaging, such as Dual Energy X-ray Absorptiometry (DEXA) and computed tomography (CT) scanners. The microstructural features are presented in the form of data based on scanning results and are also assessed and/or organized in terms of age, gender, race, pathology, clinical history, and other patient population parameters. The template can be used to assess bone quality, predict the likelihood of bone fracture, and evaluate prosthesis design and placement, based on an image of a corresponding subject bone, e.g. the bone of a patient.

### CROSS REFERENCE TO RELATED APPLICATIONS

This application claims the benefit of priority to U.S. Provisional Patent Application No. 60/839,312 filed Aug. 21, 2006, the disclosure of which is hereby incorporated by reference in its entirety.

### FIELD OF THE INVENTION

The present invention relates to the preparation and use of novel bone templates that can be prepared using a comprehensive approach to observing microstructural features of bone, including trabecular thickness and trabecular density. These features are assessed in regions of interest in a bone (e.g., proximal femur, distal femur, wrist, spine, etc.) as observed using digital radiographic techniques or clinical imaging, such as Dual Energy X-ray Absorptiometry (DEXA) and computed tomography (CT) scanners. The microstructural features are presented in the form of data based on scanning results and are also assessed and/or organized in terms of age, gender, race, pathology, clinical history, and other patient population parameters. The template can be used to assess bone quality, predict the likelihood of bone fracture, and evaluate prosthesis design and placement, based on an image of a corresponding subject bone, e.g. the bone of a patient.

Conceptually, each template represents an idealized or archetype bone of a particular kind or type, and is constructed by aggregating empirical data from observations, measurements, evaluations, etc. of similar bones obtained from cadavers. Analysis of these bones can be invasive and can provide information about bone structure and behavior that is not available, or not easily available, from non-invasive observations of a living bone. By matching a subject bone, such as a diseased or fractured living bone of a patient, to an appropriate bone template, predictions can be made about the structure and behavior of the subject bone, including for example the topographic strength of the bone, its response to stress and strain, the likelihood of fracture, fracture propagation, and response to a prosthetic or implant.

The present invention is especially valuable for those suffering from bone disorders, such as osteoporosis, an age-related disorder characterized by decreased bone mass and increased susceptibility to fractures, osteomalacia, a softening of the bones resulting from defective bone mineralization, or osteopenia, a condition of decreased bone mineral density that can be a precursor condition to osteoporosis.

### BACKGROUND OF THE INVENTION

Digital radiographic techniques are used to provide a quantified measurement of bone density. For example, DEXA involves a measurement of bone mineral density derived from the varying absorption of the bone of x-rays at different energies. DEXA scans provide quantitative in vivo measurement of bone mineral density (BMD). Other clinical imaging or digital radiographic techniques, such as CT scanning, may also provide measurements of bone density and can be used in connection with the present invention in the same manner as DEXA scans.

Adequacy of bone tissue for mechanical function is currently assessed on patients by either DEXA scan in vivo or by microCT on biopsies from the iliac crest. Although DEXA is considered the best osteoporotic diagnostic technique (Bahan and Kelly 1995; Ho et al., 1990), it does not provide information on trabecular deterioration, which may or may not include fractures. (Trabeculae are supporting strands of connective tissue in bone and constitute part of the framework of the bone.) Further, bone density per se does not fully describe bone tissue's quality, and in particular does not accurately predict the likelihood of bone fracture. For example, given two patients with equal DEXA scan values in the femoral neck region, one patient may suffer a fracture in this location while the other may remain fracture free for life. Previously, regional differences in trabecular thickness in the proximal femur utilizing microCT and magnetic resonance imaging (MRI) have been identified. Attention to alteration of parameters that describe the microstructure of bone is required to be able to assess the quality of tissue and predict the occurrence of fractures.

Accurate assessment of fracture risk is critical to establishing the threshold for clinical intervention. (Cheng, 1997; Dequeker, 1994; Abrahamsen, 2006). Starting in the mid 1980s, the adequacy of patients' bone tissue for biomechanical function has been assessed by using DEXA to estimate BMD, a partial indicia of bone quality itself determined by many factors. (Havill, 2007). Subsequently, CT scanning, MRI and ultrasound became available as non-invasive methods to study the trabecular microstructure. Further, microCT on biopsies, usually from the iliac crest, provides information on BMD and additional microstructural parameters. (Kothari, 1998; Tothill, 1989; Mazess, 1992; Krug, 2005; Krieg, 2006). Because BMD alone is insufficient to describe the adequacy of the bone tissue, research has sought to define additional microstructural parameters that together with BMD would characterize the quality of the bone tissue so as to enable appropriate clinical intervention.

The proximal femur (e.g. Ulrich, 1999; Seeman, 2006) has been a focus of research in this sense in order to target intervention (Lieberman, 1995) to avoid formation of so-called fragility fractures responsible for significant morbidity and mortality in elderly patients. (Brenneman, 2006) Such fractures result from reduction in the amount, quality and architecture of bone. After the vertebral bodies, the proximal femur appears to be the anatomical site most likely to experience non-traumatic fractures (Wasnich, 1996; Patel, 2006; Pulkkinen, 2006; Somay-Rendu, 2007). The fractures at the proximal femur, which occur primarily at the location of the femoral neck and greater trochanter (Zuckerman, 1996; Tsangari, 2006), are yet to be explained in terms of ultra- and micro-structural parameters.

The microstructural specifications pertinent to bone quality are the object of abundant inquiry. Notable initial results of this inquiry include assessment of the contribution of geometry and BMD to the failure load of the proximal femur in older women and men (Bousson, 2006; Pulkkinen, 2006), as well as the link between BMD and risk of hip and nonvertebral fractures. (Cummings, 2006). Also noteworthy is an attempt to measure the degree of organization of the trabecular network by the characterization of trabecular anisotropy through Fast Fourier transform on bone radiographs. (Chappard, 2005).

Since the mid 1800s (Ward, 1838; Roux, 1895; Tobin, 1955; Garden, 1961; Whitehouse and Dyson, 1974), the proximal femur's trabecular structure has been observed to differ across the femoral neck, Ward's triangle, greater trochanter, and intermediate region between epiphysis and diaphysis at any given age.

According to the invention, the proximal femur structure depends on age, selectively with respect to the site or region of the bone. The structural differences across sites in terms of age are interpreted in terms of Pauwel's assessment of stresses per site by photoelastic experiments (Pauwels, 1965). Pauwel's model shows that the stresses are greatest at the medial side of the femoral neck in the zone of the Adam's bow and the smallest stresses are found at the Ward's triangle. Moreover the model shows that the trabecular distribution tends to follow the line of action of the force distribution acting on the proximal femur. The mechanical function of trabeculae is more relevant at a high than at a low stress site. Roux in 1896 was the first to hypothesize that the trabecular thickness and the stress magnitude at the site should be correlated (Roux, 1896).

A study described in Link, et al. 2002 analyzes the whole cadaver proximal femur by DEXA scan and by high-resolution magnetic resonance imaging in an attempt to correlate bone density and structural parameters at specific sites (femoral head, neck and greater trochanter). Link, et al. claim that it is the presence and the uneven distribution of bone marrow that renders image resolution (binarization) difficult and may falsely increase the structural parameters, as demonstrated by Kuiper et al. (1996). The present inventors also believe that the low number of examined femurs per decade and lack of sex differentiation (31 specimens from 14 female and 17 male Caucasian donors ranging in age from 29 to 91 years of age) causes confusion of results. Therefore, the present invention is based on direct measurement of structural parameters in longitudinally sectioned proximal femurs obtained from cadavers, and free from bone marrow. The femurs can be categorized by age, gender, pathology, etc., and it will be understood that a higher sample size, with greater correlations of similar parameters, is likely to produce more reliable or statistically significant data.

The principles of the invention can be applied to any bone of any time. The femur, and particularly the proximal femur, is a preferred embodiment, because this bone is a common source of problems for patients, and has been extensively studied.

### SUMMARY OF THE INVENTION

The present invention relates to the preparation of a template that can be utilized by imaging technologies as an analytical aid to assessing the quality and condition of a subject bone. The template is based on specifying and evaluating microstructural parameters that may be responsible for fracture, specifically regional patterns of trabeculae that either thin down or decrease in number or both with age. To assess such parameters, the invention uses a method of analysis that permits direct observation of bone microstructure under high resolution. In particular, the invention analyzes two microstructural parameters—trabecular thickness and trabecular density—in human bone, such as the proximal or distal femur.

In one embodiment, the invention relates to a method of creating a bone evaluation template, comprising: (a) identifying one or more regions of a subject bone; (b) obtaining at least one control bone corresponding to the subject bone and having at least one selected region corresponding to a selected region of the subject bone; (c) obtaining a digitally scanned image of a trabecular structure of each selected region of each control bone; (d) identifying one or more trabecular families within each selected region of each control bone; (e) identifying one or more microstructural sites based on the trabecular families; (f) determining a percent trabecular density and an average trabecular thickness in each of the microstructural sites; (g) subdividing each selected region of each control bone into sections of related trabecular structure based on the trabecular families, trabecular densities, and trabecular thicknesses; and (h) based on the sections of related trabecular structure, creating a bone evaluation template for evaluating the subject bone. Additionally, the method may further include the steps of displaying the subdivided sections to form a template, and superimposing the template onto a digitally scanned image of the subject bone.

According to the invention, a template is created to display relatively homogeneous subdivisions of the particular bone or regions thereof, which undergo similar degradation in terms of, for example, presence/absence of osteopenia/osteoporosis and age. As noted above, the level of similarity within a subdivided section that is suitable to be considered “relatively homogeneous” can be determined on a case-by-case basis in consideration of the level of variation seen in the largest microstructural site assessed in cadaveric samples within the same age group. For example, in the proximal femurs described in Example 1, “relatively homogeneous” refers to microstructural sites having values for average trabecular thickness that vary within a standard deviation of ± about 30 μm in each donor age group, and having values for percent trabecular density that vary within a standard deviation of ±10%. As another example, in the distal femurs described in Example 2, “relatively homogeneous” refers to microstructural sites having values for average trabecular thickness that vary within a standard deviation of ± about 12 μm in each donor age group, and having values for percent trabecular density that vary within a standard deviation of ±2%.

In another embodiment, the present invention relates to methods of using the templates described herein in combination with digital radiographic and clinical imaging techniques. For example, an appropriately corresponding template may be input into a DEXA scanner (or other type of scanning equipment) such that it may be automatically superimposed onto the image of a patient's bone following use of the DEXA scanner to capture a scanned image of the patient's bone. Use of a template made in accordance with the present invention allows for a more realistic and accurate depiction of the trabecular structure of the patient's bone.

In a further embodiment, the present invention can be used in a method for evaluating bone quality in connection with making medical determinations and decisions regarding bone reconstruction and prostheses. For example, accurately identifying the weaker versus stronger areas of trabecular microstructure within the region of interest in a patient's bone can provide critical information that would allow better placement of bone implants (e.g., titanium screws in knee replacement surgery) and better overall medical treatment. Similarly, using the template of the present invention would provide a clearer representation of the rarefaction of trabecular microstructure than what is currently available, and would thus allow for better placement and attachment of prostheses to subject bones.

### BRIEF DESCRIPTION OF THE DRAWINGS

FIG. 1. (A) This schematic shows the position of the longitudinal cut along the axis (An) of the femoral neck through the center of the femoral head with respect to the medial-lateral (ML) axis. The arrow points to the anterior aspect. (B) The trabeculae are exposed for halved proximal femur #8 at 16% reduction. (C) The right family (designated by straight line at the left of the image of FIG. 1C) of trabeculae runs from the medial metaphysis through the neck and fans out in the head; the bent family (designated by curved line in the middle of FIG. 1C) runs from the lateral metaphysis through the neck and crosses with the right family in the central head; the medial family (designated by straight line at the right of FIG. 1C) crosses the bent family at an acute angle to enter the medial metaphysis; and the greater trochanter family (designated by perpendicular crossed lines at the right of FIG. 1C). (Ward, 1838; Singh, 1970). The location and direction of the lines shown in FIG. 1C indicate the location and directional orientation of the respective family.

FIG. 2. The trabecular thickness was labeled with segments perpendicular to the walls of each trabeculae located on the plane of the cut (14×).

FIG. 3. The trabecular density was measured as the ratio of the number of hits (white dots) and the total number of hits and misses (white and black dots) multiplied by 100 (9×).

FIG. 4. (A) This novel subdivision of the proximal femur follows the distributions of the trabecular families (FIG. 1C) in relatively homogeneous microstructural sites: central head (ch), epiphyseal head (eh), neck (ne), greater trochanter (tr), intermediate region (int) and Ward's triangle (wa). (B) The subdivision of the proximal femur conventionally used for DEXA and CT scans: head (h), neck (ne), greater trochanter (tr), intermediate region (int) and Ward's triangle (wa).

FIG. 5. This diagram shows the patterns of (A) mean trabecular thickness (Tr.Th in μm), (B) the trabecular density (Tr.Dn in %), (C) the total bone density (BMD in g/cm²) by DEXA with the exclusion of the Ward's triangle, and (D) the associated t-score in terms of age. The filled squares denote the femurs with normal BMD, while the empty squares denote the femurs affected by either osteopenia or osteoporosis.

FIG. 6. The sites considered are the central head (ch), epiphyseal head (eh), neck (ne), greater trochanter (tr), intermediate region (int) and Ward's triangle (wa) for each donor group (VI, VII, VIIo). Filled (vs empty) circle or triangle indicates significant difference with preceding value within each pattern of ch, eh, ne, tr, int, wa. Triangle (vs circle) indicates significant difference with preceding value within each of VI, VII, VIIo. (A) Mean (plotted) and standard deviation (tabled) of trabecular thickness (measured in μm) were computed on an average of 1976 measurements for each of the nine proximal femurs. (B) The percentage (plotted) and standard deviation (tabled) of trabecular density was computed on an average of 3,151 for each of the nine femurs. The segmented patterns in (A) and (B) emphasizes differences among neck, trochanter and intermediate.

FIG. 7. The diagrams compare the percent difference between the VII and VIIo groups of trabecular thickness (A) and of trabecular density (B) for the newly (new) proposed regions and for the currently (curr) employed regions by DEXA and CT scanners. Significant differences (p<0.01) relative to trabecular thickness were found at the trochanter (tr) and relative to trabecular density at the trochanter (tr) and at the intermediate (int) between new and conventional regions. The trabecular structure of the greater trochanter usually employed by the DEXA scan (C) differs above and below the line (D) (5×). The intermediate region between epiphysis and diaphysis (E) differs above and below the lines (F) closer to the neck, because the bent and medial families intersect, and towards the greater trochanter family (FIG. 1c) (2×).

FIG. 8. Graphs representing data relative to each femur, wherein such data was grouped as shown in Table 1 (A) and Table 2 (B) into VI (sixth decade with normal bone density); VII (seventh decade with normal bone density) and VIIo (seventh decade with low bone density). The sites considered are the central head (ch), epiphyseal head (eh), neck (ne), greater trochanter (tr), intermediate region (int), and Ward's triangle (wa) for each donor group. Average of trabecular thickness was computed on an average of 1976 measurements for each of the nine proximal femurs. Percentage trabecular density was computed on an average of 3,151 for each of the nine proximal femurs.

FIG. 9. Image of the exposed trabecular structure of a female post-menopausal distal femur obtained using an HP Scanjet 4890 desktop scanner set to a resolution of 1000 dpi.

FIG. 10. Image showing subdivision of the distal femur that follows the distributions of the trabeculae in relatively homogeneous microstructural sites: epiphysel condyle (E), condyle (C), and middle (M) region between epiphysel condyle and condyle.

FIG. 11. Specifications of degradation of the trabecular tissue in distal femurs of post-menopausal women. Diagrams show the patterns of (A) mean trabecular thickness (Tr.Th in μm), (B) the trabecular density (Tr.Dn in %), (C) the BMD, and (D) t-score, all in terms of age. The filled squares denote the femurs with normal BMD, while the empty squares denote the femurs affected by either osteopenia or osteoporosis.

FIG. 12. Diagrams showing decrease of trabecular thickness (A) and trabecular density (B) occurring at rates that depend on the specific site. The sites considered are the epiphysel condyle (E), condyle (C), and middle (M) region for each donor group (VI, VII, VIIeo). A filled circle or triangle indicates significant difference with preceding value within each of E, C, M. A triangle (as opposed to a circle) indicates significant difference with preceding value within each of VI, VII, VIIeo. (A) Mean (plotted) and standard deviation (tabled) of trabecular thickness (measured in μm) were computed based on an average of measurements for each of the nine distal femurs. (B) The percentage (plotted) and standard deviation (tabled) of trabecular density was computed based on an average for each of the nine femurs.

### DEFINITIONS

A selected region of a control bone (e.g. a cadaveric bone), or a region of a bone template herein, can be said to “correspond” to a selected region of a subject bone if both bones have at least one structural and/or mechanical feature in common. For example, non-limiting examples of such features include that the cadaveric donor bone and the subject bone may be: in the same decade of age (e.g., age 50-59), of the same gender, of the same type (e.g., proximal femur, distal femur, wrist, spine, iliac crest, etc.), diagnosed with (or having shown signs of) the same pathology or clinical history (e.g., osteoporosis, osteomalacia, osteopenia, etc.), of the same race (e.g., Caucasian), etc.

The subject bone can be any type of bone (e.g., proximal femur, distal femur, wrist, spine, etc.), but preferably is one having a higher likelihood to sustain non-traumatic fracture. The subject is a vertebrate, preferably a mammal, more preferably a human. The methodology of the present invention allows formulation of a template for any bone. The subject bone is from a different person than the control bone, and the subject person may be from a living person.

“Digitally scanned images” refers to in vivo images of a bone in a living subject (e.g., a human patient), a bone in a non-living donor, or a biopsy taken therefrom, and may be obtained invasively or non-invasively by conventional digital imaging techniques, such as digital radiographic and clinical imaging techniques (e.g., DEXA, CT, MicroCT, MRI, ultrasound, and the like), which provide assessments of bone density, such as calcium content. (Cann, 1988; Somay-Rendu, 2007). “Digitally scanned images” also refers to images of cadaveric bone, and may be obtained by any optical technique that is capable of taking high resolution digital images (e.g., regular light microscopy, HP Scanjet 4890 desktop scanner). “Optical microscopy images” generally includes all types of digital scans of cut bones, including those obtained by a DEXA scan of a donor bone.

“Trabecular families” refer to groups of trabeculae that show similar directional patterns and distributions (e.g., right, bent, medial, and greater trochanter families in a proximal femur) when viewed microscopically. The directional locations of the trabecular families can be used to define the microstructural sites. For instance, Ward's triangle in a proximal femur is located within the right, bent, and medial families.

“Microstructural sites” refer to any specified or predetermined locations in a bone. Preferably, such sites are regions of bone spanned by one or more trabecular families so that the trabecular family or families in each microstructural site is/are consistent throughout each site. The “microstructural sites” can serve as “landmarks” of bone architecture. For example, in proximal femurs, “microstructural sites” include the central head, epiphyseal head, femoral neck, greater trochanter, intermediate region, and Ward's triangle. (Looker, 1995). In distal femurs, “microstructural sites” include the epicondyle, condyle and middle region.

Note that as used herein “a selected region of a bone” encompasses one or more “Trabecular families” and one or more “Microstructural sites.”

The term “relatively homogeneous trabecular structure” refers to subdivided sections or volumes of bone in which microstructural sites have similar trabecular density and trabecular thickness. It will be apparent to the person of ordinary skill in the art that the range or degree of similarity may vary as desired, and can be determined or selected according to the particular use or application at hand. Preferably, similarity of structure is evaluated within each site and preferably among donor bones grouped by decade, e.g. within a 10 year range. Similarity of structure may also be evaluated among bones of the same gender and/or among those having the same pathology or other common feature.

### DETAILED DESCRIPTION OF THE INVENTION

The present invention relates to the preparation of a template that can be utilized by imaging technologies as an analytical aid to assessing the quality and condition of a subject bone. The template is based on specifying and evaluating microstructural parameters that may be responsible for fracture, specifically regional patterns of trabeculae that either thin down or decrease in number or both with age. To assess such parameters, the invention uses a method of analysis that permits direct observation of bone microstructure under high resolution. In particular, the invention analyzes two microstructural parameters—trabecular thickness and trabecular density—in human bone, such as the proximal or distal femur.

Trabecular thickness and trabecular density can be assessed separately in selected regions by semi-automatic histomorphometry. (Parfitt, 1983). According to the invention, the parametric values for each of trabecular thickness and trabecular density vary among different regions of the bone at any given age. Furthermore, trabecular thickness and trabecular density vary independently from each other in terms of age. Changes in terms of age for each parameter depend for example on the presence or absence of osteoporosis. Microstructural patterns found and described herein may be readily adapted by persons of ordinary skill in the art to defined regions of interest for clinical imaging (e.g., DEXA scan use) as applied to any bone type in order to acquire additional information on a patient's bone quality.

The present invention results from investigations of the microstructure of bone, particularly cancellous bone (e.g., at the proximal or distal femur). These investigations allow for definitions of microstructurally meaningful regions of the bone (i.e. within the bone volume) that offer the prospect of improved assessment of fracture risk through clinical imaging technology. A digitally scanned image of the plane of the section of each studied bone, along with labeling of trabeculae can be used to examine the morphometry of the trabeculae. This can be done manually or with the aid of a computer. Mean trabecular thickness and trabecular density were determined, and were found to vary with age and independently by site. The variation of mean trabecular thickness and trabecular density differed between normal and osteopenic/osteoporotic conditions. Trabeculae at the more mechanically-stimulated sites degrade more slowly with age under normal conditions, consistent with normal fracture risk, whereas osteopenic/osteoporotic conditions tend to accelerate degradation of trabeculae. Accordingly, the invention employs a template that divides the proximal or distal bone into regions of relatively homogeneous microstructure. This provides information on the trabecular changes due to aging and presence/absence of osteopenia/osteoporosis that are relevant to assessment of fracture risk.

The present invention is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled.

The data analysis described in the following examples refers to 40× images of the medial longitudinal section of female donors aged 50-70 analyzed using vector graphic software (e.g., XaraX1 available from XaraX Co) and analysis software (e.g., MetaMorph available from Universal Imaging Co. These examples relate to studies of donors of selected medical history in order to focus on post-menopausal women with either normal bone density (as assessed by DEXA scan) or osteoporosis, and free of metabolic agents that could further alter the bone structure. The donated femurs, classified by bone mineral density, of post-menopausal women in the sixth and seventh decade of life were sectioned through the center of the femoral head and along the femoral neck axis. However, the methods and analysis described herein may be extended from femurs to other types of bone from corresponding donors with specific medical histories, and the collective data can be used to create a data bank correlating medical histories, trabecular microstructure, and DEXA scan values. Clinically, DEXA scan values will thus assume a greater diagnostic value.

The results of the studies described in these examples add meaning to the diagnosis of osteoporosis by DEXA scan by uncoupling two fracture risk factors: lower than normal bone density and trabecular thickness in terms of age and sex. Additionally, the scope of the present invention can include a larger female specimen pool in terms of decades and number of specimens per decade or could also include a male specimen. The invention and template herein can also be adapted to include other applications, such as:
thin sections of isolated trabecular sites embedded in resin could be analyzed under circularly polarizing light for indication of collagen bundle orientation patterns, which are expected to follow the maximum stress trajectories as is the case of compact bone (Ascenzi A., 1988);
trabecular specimens of specific collagen bundle orientation and degree of calcification from the various sites could be isolated and mechanically tested to assess each site's mechanical properties through site modeling; since collagen bundle orientation and degree of calcification distributions are expected to determine the mechanical properties of trabeculae as they do for compact bone's osteons (Ascenzi M.-G. et al., 2000). Current isolated trabecular specimens are undifferentiated with respect to collagen bundle orientation and degree of calcification (Bini et al., 2002); and
trabecular thickness of the proximal or distal femur (or other bone) affected by other pathologies, which are very different from osteoporosis but reveal themselves through an abnormal DEXA scan pattern, could be investigated along the lines of the foregoing study. One example is cerebral palsy, the most common childhood disability in the United States.

Further, the present invention may contribute to the understanding of osteoporotic medications' effects on the bone tissue by providing structural information at the various sites on which the medications operate.

### EXAMPLE 1

### Study Of Female Post-Menopausal Proximal Femurs

This example describes a study of the proximal femur that first seeks to clarify trabecular changes due to the process of aging by analyzing the two trabecular parameters of trabecular thickness and trabecular density in female donors in the sixth and seventh decade of life using histomorphometry based on direct microscopic observation. Second, the two trabecular parameters are correlated with BMD information obtained by DEXA and were found to be independent. With age, mean trabecular thickness and trabecular density were found to vary independently from each other, but dependently by site. Further, the variation of mean trabecular thickness and trabecular density differed between normal and osteopenic/osteoporotic conditions.

The results of this study provide insight into the alterations in the cancellous bone microstructure at the proximal femur, a clinically-relevant skeletal site, notwithstanding the limitation that this initial study was conducted on two-dimensional data. The trabeculae at the more mechanically-stimulated sites (femoral neck and trochanter) were found to degrade more slowly with age under normal conditions, consistent with the higher mechanical stresses present at such locations. (Van Rietbergen, 2003). However, osteopenic and osteoporotic conditions were found to accelerate the degradation of trabeculae with a marked mechanical function. A new template that reflects microstructurally meaningful regions is proposed, which can improve the assessment of fracture risk by allowing more effective aggregation and interpretation of BMD data collectable through currently-available clinical imaging technology.

Materials And Methods

Study Design: The cadaver proximal femur was subjected to DEXA scan to assess bone density at the femoral neck, Ward's triangle, great trochanter, and intermediate region between epiphysis and diaphysis measurements within each square. After longitudinal sectioning and bone marrow removal, bone density and trabecular thickness was assessed at each site (femoral neck, Ward's triangle, great trochanter, and an intermediate region between epiphysis and diaphysis) by histomorphometric methods.

Study procedures: A tissue procurement agency provided the bone tissue for research. The bone tissue exclusions/requirements for each donor, including acceptable conditions in the donor population studied, were:
Exposure to chemotherapy was not acceptable, unless it occurred more than 10 years prior to death.
Radiation was acceptable as long as it is not localized to the hip joint and leg.
Cancer was acceptable as long as bone metastases can be positively excluded.
Hepatitis A+ or B+ was acceptable.
Donors with Hep C, HIV, diabetes, kidney abnormal function, kidney failure, chronic liver disease, osteomalacia, osteopetrosis, pathologies of the hip joint and/or knee, Paget disease, partial hip implants, and knee implants were avoided.
Bacteria, such as Clostridium botulinum, was acceptable. A localized infection (e.g. pharyngitis, cellulitis, pneumonia) that is suggested by high fever, as the only symptom, was acceptable. Sepsis, intended as systemic spread of the offending organism, was not acceptable.
No drugs that alter bone structure were acceptable. In particular, no osteoporotic drugs such as alendronate (fosamax), risedronate (actonel), raloxifene (evista), calcitonin (miacalcin), estrogen, prednisone, or parathyroid hormone were acceptable.
Donors having long-term (more than 6-8 weeks) bed rest or ventilator time were not acceptable.

Tissue Constraints: Procure single, normal femur from autopsy within 8 hours; wash in sterile water, dry with sterile tissue (paper or cloth), and freeze immediately; and maintain at −80C.

Shipping Constraints: Ship on plenty of dry ice by overnight delivery.

Preparation and selection of specimens: The International Institute for the Advancement of Medicine, the Indiana Organ Procurement Organization, the Musculoskeletal Transplant Foundation, The National Disease Research Interchange, and ScienceCare Anatomical provided the human femurs used for this study. The clinical history of each donor was confirmed as free from medical therapies that could have altered the bone tissue and metabolic pathologies, except for post-menopausal osteopenia and osteoporosis. After removal and defleshing, each femur was washed in water and allowed to dry at room temperature, and histology of the trabecular femoral tissue assessed either presence or absence of osteomalacia through thickness of osteoid border. Femurs of nine Caucasian female donors (one from each) ranging in age from 52 to 70 and free from osteomalacia, were employed for this study and represented biological variation of density and microstructure.

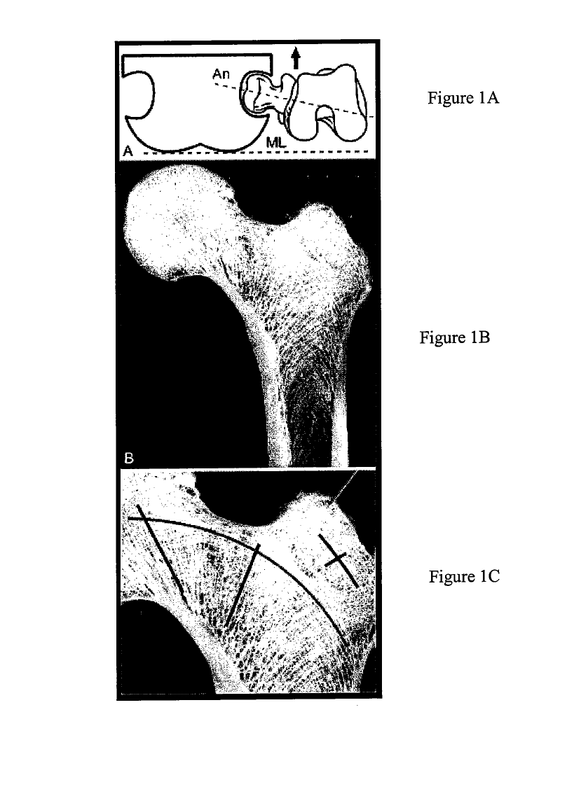

To assess BMD, each proximal femur was embedded in rice bags for standard simulation of soft tissue (Hologic, 1996) and positioned under a DEXA scanner Delphi A (Hologic Inc.) equivalently to the femur of a patient lying supine under the scanner. Each proximal femur was then sectioned longitudinally through the center of the femoral head along the femoral neck axis (FIG. 1A) by means of a high-precision sectioning saw (Harrington Tool Co., Michigan). The bone marrow closer to the cut surface of each half proximal femur was removed by enzymatic digestion enhanced by a solution of water and Tergazyme (Alconox, Inc). (Boyde, 1984). Areal bone density readings (g/cm²) with a resolution of 1.5 mm were obtained at femoral neck, Ward's triangle, greater trochanter, and intermediate region between epiphysis and diaphysis. The data was tabulated per site and decade of age.

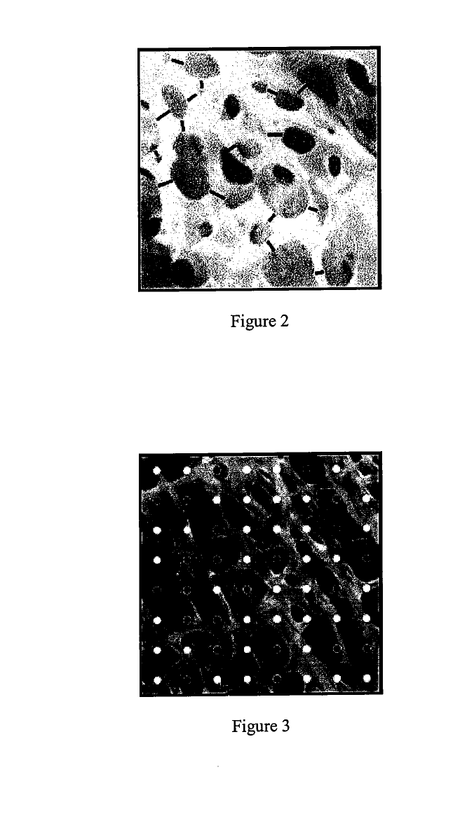

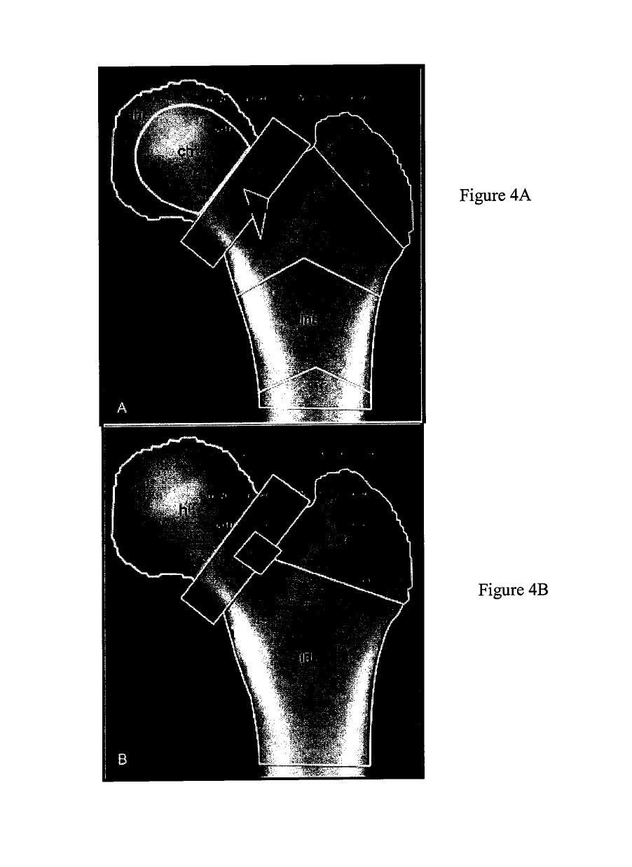

Imaging and morphometry: Images of the exposed trabecular structure were obtained using an HP Scanjet 4890 desktop scanner set to a resolution of 1000 dpi (FIG. 1B). The images were imported into the graphic software XaraX1 (XaraX Co) and analyzed at 40×. The morphometric analysis of each image referred to the trabecular structure (FIG. 1C) that appeared on the focal plane. (Scolamacchia, 1999). Specifically, on each trabecular rod (Odgaard, 1997) throughout the image of the proximal femur section, a line segment was manually drawn perpendicularly to the trabecular walls with a minimum of 200 μm between subsequent segments along a non-branching trabecula (FIG. 2). Semi-automatic measuring of segments was performed with the image analysis software MetaMorph (Universal Imaging Co). Trabecular thickness was measured for trabeculae on focus (Odgaard, 2001). The measurement error was at most equal to ±10 μm and comparable to current studies (Link et al., 2002). A 900 μm grid was superimposed electronically to each image to assess trabecular density. A white dot placed at a given intersection point of the grid denoted a hit, i.e. the presence of a trabecula (Parfitt, 1983), while a black dot denoted a miss, i.e. lack of trabecula at such intersection point (FIG. 3). Semi-automatic counting of hits and misses separately in the desired region of the proximal femoral image was performed with MetaMorph. The trabecular density was computed using the ratio of the number of hits to the total number of hits and misses, within the region of interest, multiplied by one-hundred. (Parfitt, 1983) The sites of epiphyseal head, central head, neck, Ward's triangle, greater trochanter delineated above the insertion of the vastus lateralis, and the intermediate region between the epiphysis and diaphysis below the insertion of the vastus intermedius (FIG. 4A) were chosen for the analysis. The discussion below addresses the choice of definition of trochanter and intermediate regions distinct from the regions with the same name conventionally used for DEXA and CT scans (FIG. 4B). The femurs were divided into three groups of three femurs each by decade of age and total t-score: VI, the sixth decade with t-score between −1 and 1; VII, the seventh decade with t-score between −1 and 1; and VIIo, the seventh decade with t-score lower than −1.

Statistical analysis: Intra and inter-observer error were assessed by having two observers independently label the trabeculae for trabecular thickness and trabecular density at the six chosen sites, with two repetitions each. Both intra- and inter-observer error were calculated at less than 5% by comparisons of means for trabecular thickness and of percentages for trabecular density. The student t-test was run on the trabecular thickness data, and the statistical test of inference for proportions was run from the trabecular densities, in each instance with the level of significance set at 0.05.

At least nine normal and osteoporotic, female cadaver femurs per each decade of age, between 50 and 70, were investigated and represented the biological variation of density and microstructure from a preliminary study following the observations of Lozupone and Favia (Lozupone and Favia, 1990). The means for the trabecular thickness and bone density assessed histomorphometrically were compared by t-test (using ANOVA) and among different sites for each fixed decade of age and among different age groups for each specific site, for normal and osteoporotic femurs separately. ANOVA was used to establish differences between normal and osteoporotic femoral sites for bone density assessed histomorphometrically and by DEXA, separately, and trabecular thickness assessed histomorphometrically. The bone density assessed histomorphometrically was expected to correlate well to the bone density assessed by DEXA in terms of site and decade of age, for normal and osteoporotic femurs, separately. The underlying assumption of the correlation is that the trabecular mass density is constant across the 3-dimensional sites. The number obtained by multiplying the bone density assessed histomorphometrically by the trabecular mass density was expected to be larger than the bone density assessed by DEXA. This is because the bone density assessed histomorphometrically includes volumes occupied by components other than mineral, such as water. Patterns of statistically significant changes in bone density and trabecular thickness throughout the 50-70 age range were mapped out for the four sites of normal and osteoporotic bone, separately.

Results

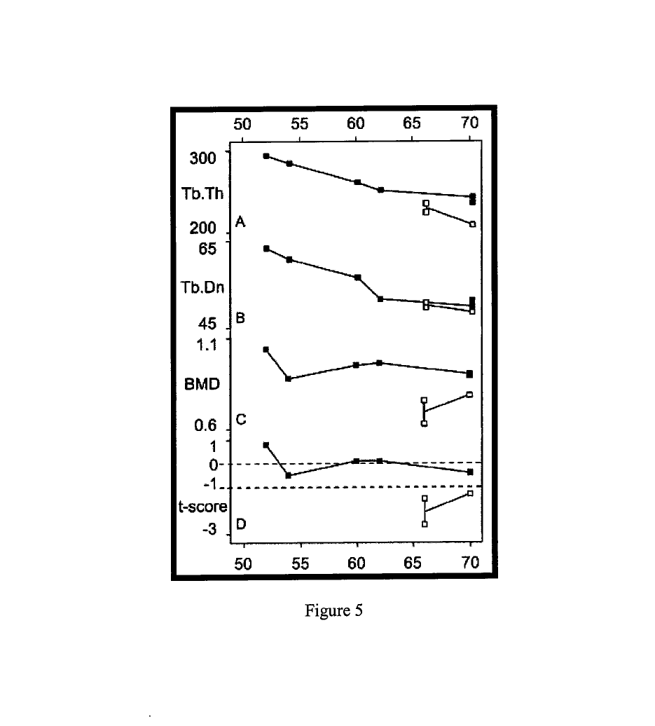

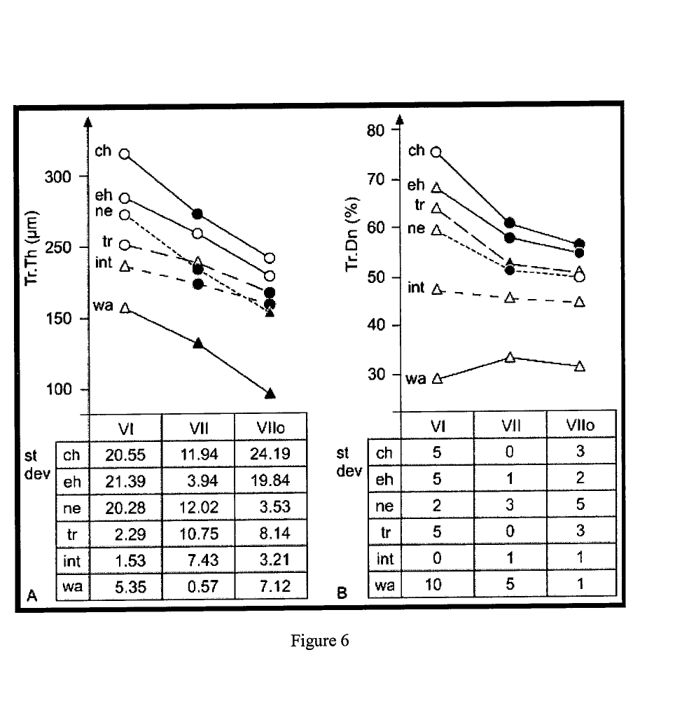

This study addresses the specifications of degradation of the trabecular tissue in the proximal femur of post-menopausal women. Trabecular thickness and trabecular density decrease with age at different rates (FIGS. 5A and 5B), following the generally decreasing ranking of BMD (FIG. 5C) and t-score (FIG. 5D). Such decrease occurs at rates that depend on the specific site (FIG. 6). Specifically, under normal conditions, during the seventh decade (VII group), the mean trabecular thickness (FIG. 6A) in the trochanter of specimens with normal bone density becomes larger than the mean trabecular thickness at the neck, therefore altering the decreasing ranking observed in the sixth decade (VI group) of central head, epiphyseal head, femoral neck, greater trochanter, intermediate region and Ward's triangle, into the ranking of central head, epiphyseal head, greater trochanter, femoral neck, intermediate region and Ward's triangle. For the seventh decade specimens affected by osteopenia/osteoporosis (VIIO group), the ranking observed for the normal seventh decade (VII group) remains unaltered except that the relative extent of degradation in the intermediate and neck regions is reversed.

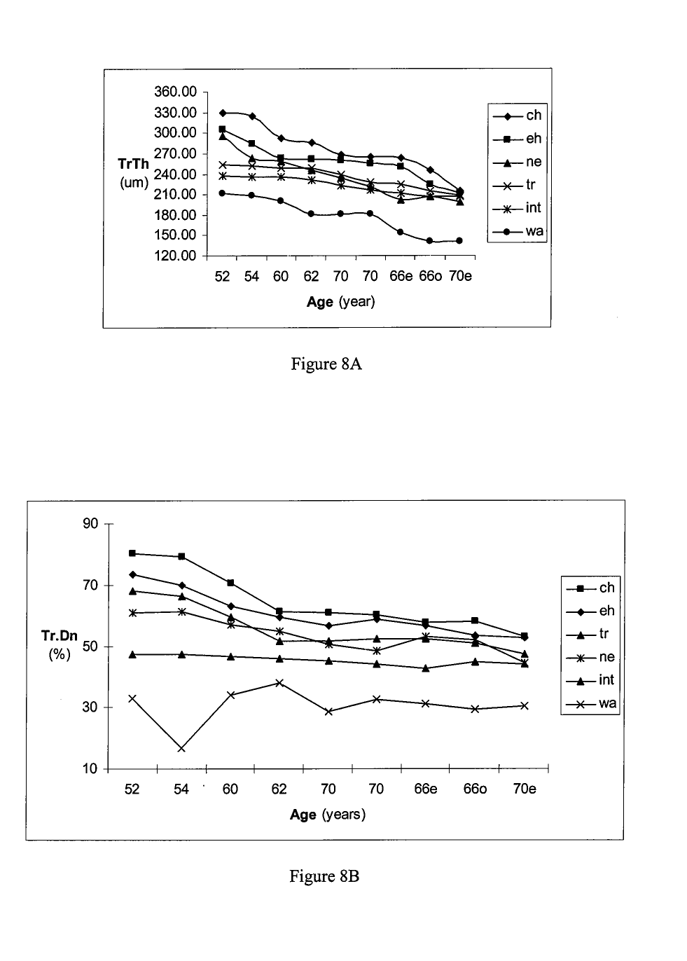

The results of the analysis of trabecular thickness are shown in Table 1 below, and in FIG. 8A.

TABLE 1
Trabecular Thickness (μm)

| | | Central | Epiphyseal | Femoral | Greater | Intermediate | Ward's |
|---|---|---|---|---|---|---|---|
| Age | T-score | Head | Head | Neck | Trochanter | Region | Triangle |
| 52 | 0.8 | 329.08 | 305.85 | 296.20 | 253.34 | 237.91 | 211.06 |
| | | 90.71 | 83.25 | 93.20 | 70.81 | 62.65 | 76.22 |
| 54 | −0.5 | 325.33 | 284.78v | 263.25v | 252.38 | 235.29 | 208.02 |
| | | 90.26 | 76.13 | 96.60 | 64.41 | 61.98 | 43.85 |
| 60 | 0.1 | 291.76 | 263.07v | 259.25 | 248.99 | 235.25 | 200.66 |
| | | 93.67 | 85.65 | 85.45 | 94.96 | 63.04 | 59.92 |
| 62 | 0.1 | 286.44 | 262.19 | 244.99 | 248.89 | 231.21 | 181.33 |
| | | 104.19 | 94.20 | 82.00 | 101.53 | 138.35 | 49.67 |
| 70 | −0.4 | 267.61 | 260.48 | 234.51 | 239.40 | 222.98 | 181.84 |
| | | 81.64 | 88.58 | 81.33 | 61.70 | 62.57 | 29.72 |
| 70 | −0.4 | 264.31 | 254.68 | 221.01 | 227.44 | 216.37 | 180.70 |
| | | 82.81 | 72.44 | 72.23 | 76.76 | 68.22 | 44.77 |
| 66 | −1.5 | 263.22 | 250.21 | 202.45v | 225.34 | 212.22 | 153.77v |
| | | 89.91 | 105.00 | 64.57 | 77.78 | 69.19 | 43.98 |
| 66 | −2.6 | 244.96 | 224.06v | 206.23 | 215.04 | 207.31 | 141.48 |
| | | 81.32 | 63.50 | 73.81 | 49.25 | 57.69 | 63.96 |
| 70 | −1.3 | 215.30 | 211.29v | 199.17 | 209.28 | 206.20 | 141.38 |
| | | 82.96 | 68.35 | 57.47 | 66.82 | 53.17 | 40.45 |

Standard deviation is shown in bold under the mean entries.

The trabecular density (FIG. 6B) decreases along the ranking of central head, epiphyseal head, greater trochanter, femoral neck, intermediate region and Ward's triangle for each of the sixth and seventh decades, under both normal and osteopenic/osteoporotic conditions (all VI, VII, VIIo groups). Note the difference in patterns between trabecular thickness and trabecular density among neck, trochanter and intermediate (dashed lines in FIGS. 6A and 6B).

The results of the analysis of trabecular density are shown in Table 2 below, and in FIG. 8B.

TABLE 2
Trabecular Density (%)

| | | Central | Epiphyseal | Greater | Femoral | Intermediate | Ward's |
|---|---|---|---|---|---|---|---|
| Age | T-score | Head | Head | Trochanter | Neck | Region | Triangle |
| 52 | 0.8 | 80 | 73 | 68 | 61 | 47 | 33 |
| 54 | −0.5 | 79 | 70 | 66 | 61 | 47 | 17 |
| 60 | 0.1 | 70 | 63 | 59 | 57 | 47 | 34 |
| 62 | 0.1 | 61 | 59 | 52 | 55 | 46 | 38 |
| 70 | −0.4 | 61 | 57 | 52 | 51 | 45 | 29 |
| 70 | −0.4 | 60 | 59 | 52 | 48 | 44 | 33 |
| 66 | −1.5 | 58 | 57 | 52 | 53 | 43 | 31 |
| 66 | −2.6 | 58 | 54 | 51 | 52 | 45 | 29 |
| 70 | −1.3 | 53 | 53 | 47 | 45 | 44 | 30 |

### DISCUSSION

The rarefaction of the trabecular microstructure that occurs with age at the proximal femur was here investigated by site through measurement of trabecular thickness and trabecular density. These two parameters were observed to decrease with age at rates that differ from each other at any given site of the proximal femur in the absence of osteopenia and osteoporosis. The presence of osteopenia and osteoporosis increases such rates differently by site and by age.

The restriction of the trabecular analysis to the plane of the section arises from the focus of the study on the thickness of the trabecular rods and the incidence of trabeculae that lie parallel to the plane of the longitudinal section and appear on focus on the plane of the section. Because each longitudinal section was consistently cut through the center of the femoral head, along the femoral neck axis, the assumption of the study that allowed the comparison among femurs is that the trabecular orientation is consistent with the orientation of the cut. What justifies this assumption is the expectation that the biomechanical function of trabeculae in the distal femur be consistent with the orientation of the trabeculae by site (Koch, 1917; Tobin, 1955; Morgan et al., 2004; Patel and Murphy, 2006; Nazarian et al., 2007). Such restriction can be partially lifted by conducting a microCT scan on smaller specific sites of interest. The restriction of the analysis of the trabecular thickness to so-called rods is due to the rod and plate structure of the distal femur, and arises from the elimination of plates from this two-dimensional study because they may lie off the plane of the cut, thereby preventing accurate measurement (Parfitt et al., 1983; Odgaard, 1997; Gentzsch et al., 2003; Stauber and Muller, 2006a and 2006b). Because this is a two-dimensional study and usually plates are oriented off the plane through the femoral neck axis and through the center of the head, plates needed to be excluded from the assessment of the trabecular thickness.

### COMPARISON OF RESULTS

Comparison of Invention Results with Previous Studies:

Previous studies by other investigators focused on the search for parameters that in addition to age, gender and BMD characterize bone quality; (Tsangari, 2006; Weiss, 2006; Hui, 1988; Khosla, 2006; Tsangari, 2007). Non-invasive clinical imaging techniques have been sought that allow the appraisal of such parameters in patients, and have yielded results that confirm the existence of regional differences of the trabecular parameters at different skeletal sites. (Hildebrand, 1999). Within the proximal femur, trabecular parameters were found to differ among the femoral head inferior to the fovea, superior to the fovea, adjacent to the femoral neck in the lateral aspect of the femoral head, and trochanteric region through MRI, CT, microCT and to be correlated with local BMD. (Issever, 2002; Sell, 2005; Meta, 2006). Correlation between ultrasonic parameters and BMD by CT was established (Sell, 2005), as well as a relation between bone quantitative ultrasound results and fractures. (Marin, 2006).

The mean trabecular thickness of this study is in general larger than that found by others (Stauber, 2006; Link, 2003; Bauer, 2006), probably due to its reference to rods in two-dimensions while the other studies refer to three-dimensional rods and plates. For the trabecular density, there is no direct comparison with a specific automated three-dimensional parameter. Nevertheless, the trabecular separation for females aged 70-90 (Bauer, 2006) with a ranking of femoral neck, greater trochanter and head, which is inverse to the present trabecular density ranking of head, greater trochanter and neck relative females aged 52-70, suggests that the observed ranking described herein maintains at older age.

Comparison of Invention Results and DEXA and CT Technologies

The trabecular thickness and trabecular density were observed to decrease with the generally decreasing patterns of total BMD in this study (FIG. 5). This is the case even though the measurements of BMD combine cortical and trabecular bone density and the loss of cortical thickness occurs with aging at rates that may differ (i) from cancellous rates (Leichter, 1987) and (ii) from the rates differentiated by the presence/absence of osteoporosis. (Lundeen, 2001; Chan, 2007). It should be noted here that because the BMD reading of DEXA at the intermediate region includes the compact bone component, it is in general much higher than expected from the trabecular observation. Indeed, it is the region with highest BMD by DEXA, but nonetheless one of the lowest trabecular thickness and trabecular density (FIG. 6).

Two of the sites chosen for the trabecular analysis (trochanter and intermediate) differ in definition from the sites of the same name conventionally employed for the proximal femur by DEXA and CT scanners (FIG. 4B). When the DEXA scanner was first built in the late 1980s (Lilley, 1991; Looker, 1995), the still conventionally-used definitions of the greater trochanter and the intermediate region between epiphysis and diaphysis were established because they captured a high incidence of fracture. (Fogelman, 1989).

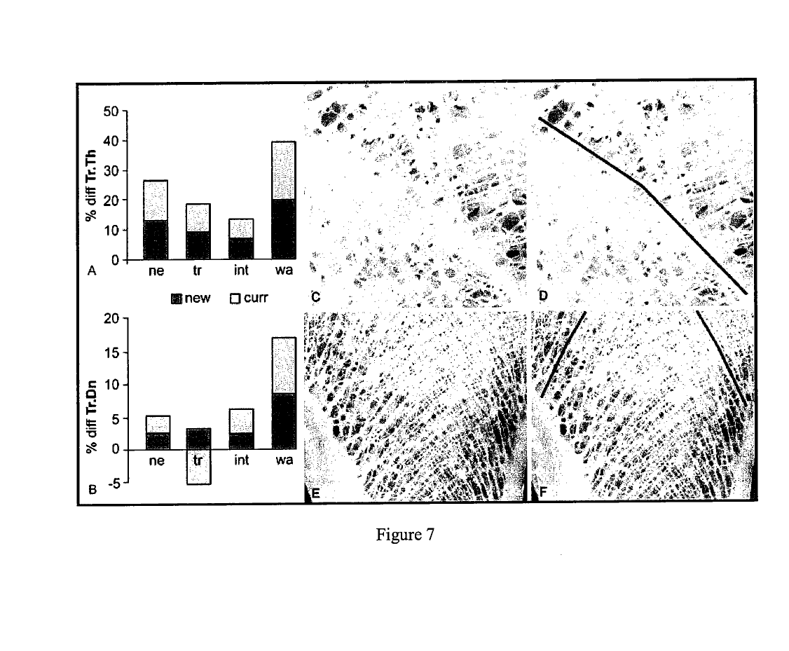

FIGS. 7A and 7B show a difference in trabecular degradation between the newly proposed trochanter and intermediate regions and the larger regions by the same name conventionally used for DEXA and CT scans, which show mixed trabecular patterns affected by a wider range of stress modalities and magnitudes. Such differences are shown to confuse the more consistent patterns of the more homogeneous regions, if lumped together. Relative to the greater trochanter and the intermediate region now conventionally employed for DEXA and CT scans, the proximal sites chosen for our trabecular analysis (FIG. 4C) restrict the greater trochanter to exclude the bent family (FIG. 1C) and the intermediate region to exclude the bent and the rarefaction of the medial family towards the Ward's triangle (FIG. 1C). This was done to have sites whose trabecular structure is relatively homogeneous and which undergoes similar degradation in terms of presence/absence of osteopenia/osteoporosis and age.

Indeed, FIG. 7C shows that the trabecular structure of the greater trochanter conventionally employed by the DEXA scan differs above and below the line (FIG. 7D). The intermediate region between epiphysis and diaphysis differs from other sites, such as closer to the greater trochanter (FIGS. 7E and 7F), which contains the medial family of trabeculae. (Lilley, 1991). The new template described herein separates the upper portion of the trochanter from the remaining lower trochanter (compare FIGS. 4A and 4B) and a central portion of the conventional intermediate region from the remaining intermediate region (compare FIGS. 4A and 4B). The trochanteric fracture that generally occurs at younger age versus the neck fracture that occurs at older age (Madore, 2006) may be due to degradation of the lower trochanter. The trochanteric region usually examined by DEXA and CT scans may provide a lower BMD (because of the lower BMD of the lower trochanter region) than for the neck. Therefore, the BMD analysis not to be limited to either the total proximal femur with the exclusion of the Ward's triangle, or to the neck region, but rather that it include separate examination of upper and lower trochanter, and of upper and lower intermediate sites, because trabecular changes may well occur differently.

Comparison of Invention Results and Mechanics of Proximalfemur

The mechanisms of trabecular thinning and reduction of number appear to occur at different rates that depend on site and age. The biomechanical make-up of the site may be responsible for such differences. (Patel, 2006) The study of bone microstructural patterns in the proximal femur and its relation to mechanical function (see, e.g., Van Rietbergen, 2003; Heller, 2001; Bergmann, 2001; Beck, 2006) dates back to Koch. (Koch, 1917). The combination of geometry and loading under normal conditions causes bending with compressive stresses on the concave side and tension on the convex side. (Van Rietbergen, 2003; Tobin, 1955). Relative to the remaining sites, the magnitude of stress is higher at the lower portion of the neck into the femoral head in compression, and at the upper portion of the greater trochanter in tension toward the upper convex portion of the proximal femur above and to include the medial family. (Boyde, 1984). At such sites, the trabeculae would undergo a substantial mechanical stimulation that would stimulate greater growth relative to the intermediate region and the Ward's triangle.

In this study, sites that undergo higher stresses show higher values of trabecular thickness and trabecular density than sites that undergo low stresses (intermediate region and Ward's triangle) (FIG. 6). While neck and trochanter do not show a significant difference in trabecular degradation during the sixth and seventh decade under normal conditions, the significant reduction in trabecular thickness at the neck and in trabecular density at both neck and trochanter between the sixth and seventh decade suggests that at the neck the trabecular reduction occurs more in thickness than in number, while in the trochanter the trabecular reduction occurs more in number than in thickness, provided that neither osteopenia or osteoporosis affects the structure. If instead osteopenia/osteoporosis affects the structure, the trabecular thinning does not further reduce the trabecular number in the VII decade. This description of trabecular degradation between neck and trochanter is consistent with a lack of trabecular structure in the trochanter also at later decades, while the neck tends to maintain microstructural presence. (Hologic, 1996)

### CONCLUSION

Additional femurs may be analyzed to ascertain the effect of macroscopic geometry, nutrition and physical activity on the evolution of the proximal femur microstructure. (Nurzenski, 2007; Kaptoge, 2007). Further, a longitudinal study on patients may ascertain the effectiveness of the newly proposed regional subdivision to extract more clinically relevant information from conventional imaging technologies. It is predicted that current imaging can actually detect the microstructural variations detected by this microstructural study. Demonstration that it can will imply redefinition of the proximal femoral template conventionally used in clinical assessment of BMD. In any event, extension of the microstructural findings of this study provides the background patterns for reference and interpretation as clinical imaging technology's ability to detect trabecular specifications in the proximal femur is further sharpened.

### EXAMPLE 2

### Study Of Female Post-Menopausal Distal Femurs

Studies of distal femoral microstructure in non-osteoporotic and osteoporotic patients are important because stress fractures are well-known overuse injuries in active people and osteoporotic patients may not be suitable for knee replacement. The femur is the fourth most common site of stress fractures (see, e.g., Matheson et al., 1987) with injury occurring in the neck, subtrochanteric, shaft, or condylar regions (Glorioso et al., 2002; Boden and Speer, 1997). Studies of site dependent bone microstructure are relevant because bone density decline due to either immobilization or lack of weight-bearing indicates site dependency (see, e.g., Giangregorio et al., 2002).

This study provides a template that allows the trabecular architectural factors that predispose a patient to sustain a nontraumatic fracture to be associated with the patient's distal femoral DEXA scan. Additionally, this study appraises trabecular thickness changes underlying the characteristic differences detected by the DEXA analysis between normal and osteoporotic proximal femurs, fractures of which are the most severe aspect of osteoporosis. The results of this study also allow those of ordinary skill in the art to develop and use more focused and effective medication therapies based on the site specific morphological information obtained by the methods disclosed herein.

Materials And Methods

Preparation and selection of specimens: The International Institute for the Advancement of Medicine, the Indiana Organ Procurement Organization, the Muscoloskeletal Transplant Foundation, The National Disease Research Interchange, and ScienceCare Anatomical provided the human femurs used for this study. The clinical history of each donor was confirmed as free from medical therapies that could have altered the bone tissue and metabolic pathologies, except for post-menopausal osteopenia and osteoporosis. After removal and defleshing, histology of the trabecular femoral tissue assessed either presence or absence of osteomalacia through thickness of osteoid border. Femurs of nine Caucasian female donors (one from each) ranging in age from 52 to 70 and free from osteomalacia, were employed for this study.

To assess bone mineral density (BMD), each femur was embedded in rice bags for standard simulation of soft tissue (Hologic, 1996) and positioned under a DEXA scanner Delphi A (Hologic Inc., Bedford, Mass.) equivalently to the femur of a patient lying supine under the scanner. Each distal femur was then sectioned longitudinally by means of a high-precision sectioning saw (Harrington Tool Co., Michigan). The bone marrow closer to the cut surface of each half distal femur was removed by enzymatic digestion enhanced by a solution of water and Tergazyme™ (Alconox, White Plains, N.Y.) as described in Boyde (1984).

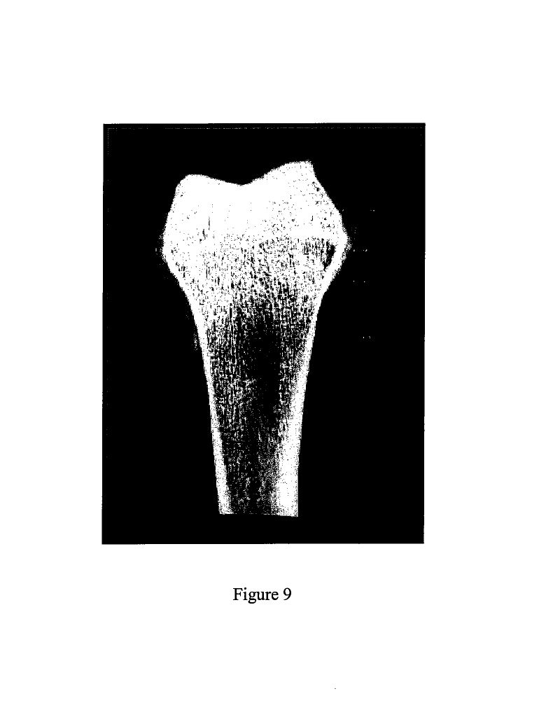

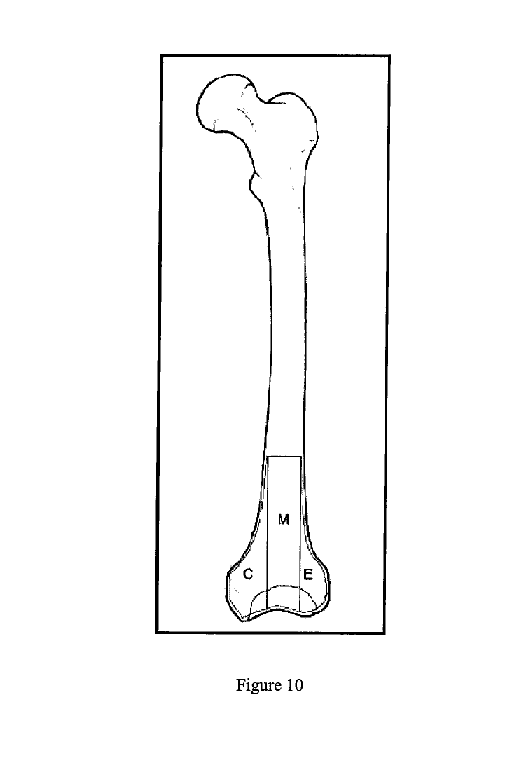

Imaging and morphometry: Images of the exposed trabecular structure were obtained using an HP Scanjet 4890 desktop scanner set to a resolution of 1000 dpi (FIG. 9). The images were imported into the graphic software XaraX1 (XaraX Co, UK) and analyzed at 40×. The morphometric analysis of each image referred to the trabecular structure that appeared on the focal plane (Scolamacchia, 1999). Specifically, on each trabecular rod (Odgaard, 1997) throughout the distal femur, a line segment was manually drawn perpendicularly to the trabecular walls with a minimum of 200 μm between subsequent segments along a non-branching trabecula. Semi-automatic measuring of segments was performed with the image analysis software MetaMorph (Universal Imaging Co). A 900 μm grid was superimposed electronically to each image to assess trabecular density. A white dot placed at a given intersection point of the grid denoted a hit or the presence of a trabecula (Parfitt, 1983), while a black dot denoted a miss or lack of trabecula at such intersection point. Semi-automatic counting of hits and misses separately in the desired region of the distal femoral image was performed with MetaMorph. The trabecular density was computed using the ratio of the number of hits to the total number of hits and misses, within the region of interest, multiplied by one-hundred (Parfitt, 1983). The sites of epiphyseal condole (e), condyle (c) and middle (m) region between epiphyseal condole and condyle (FIG. 10) were chosen for the analysis. The femurs were divided into three groups of three femurs each by decade of age and total t-score: VI, the sixth decade with t-score between −1 and 1; VII, the seventh decade with t-score between −1 and 1; and VIIeo, the seventh decade with t-score lower than −1.

Statistical analysis: Intra and inter- observer error were assessed by having two observers independently label the trabeculae for trabecular thickness and trabecular density at the three chosen sites with two repetitions each. Both intra- and inter-observer error were calculated at less than % by comparisons of means for trabecular thickness and of percentages for trabecular density. The student t-test was run on the trabecular thickness data, and the statistical test of inference for proportions was run from the trabecular densities, in each instance with the level of significance set at 0.05.

### RESULTS

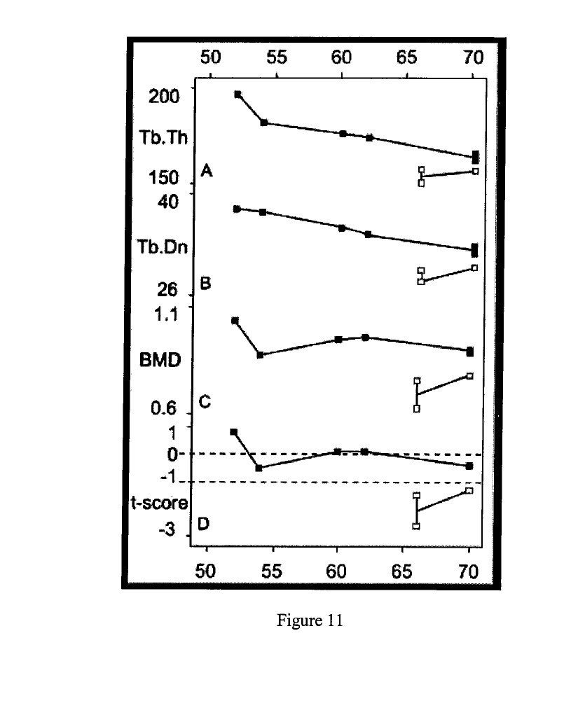

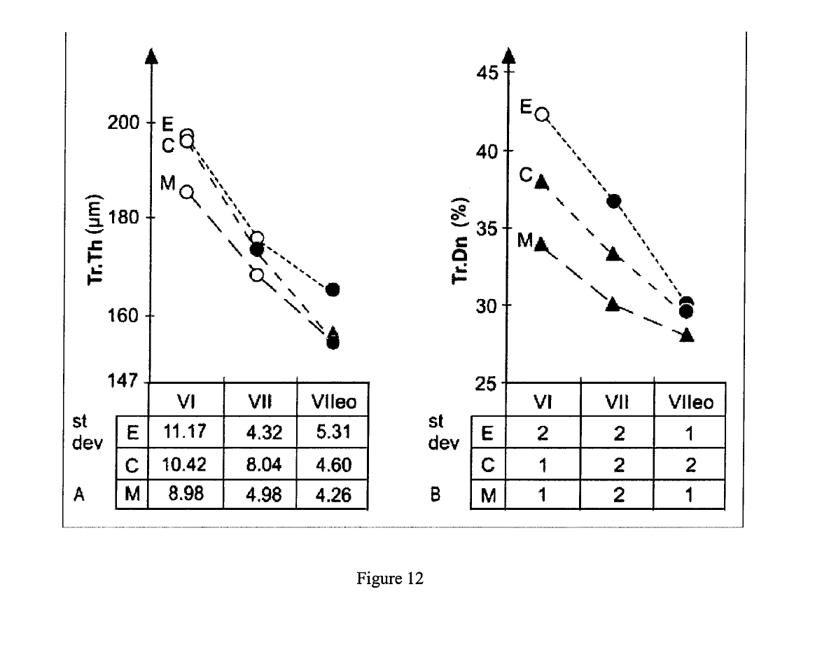

This study addresses the specifications of degradation of the trabecular tissue in the distal femur of post-menopausal women. Trabecular thickness and trabecular density decrease with age at different rates (FIGS. 11A and 11B), following the generally decreasing trend of the t-score (FIG. 11D). Such decrease occurs at rates that depend on the specific site. Specifically, the mean trabecular thickness (FIG. 12A) decreases from the highest value at the epicondyle to the lowest value at the middle region between condyle and epicondyle during the sixth and seventh decade of life, under both normal and osteopenic/osteoporotic conditions. The trabecular thickness decreases significantly at the condyle between the sixth and seventh decade during normal conditions while the difference between normal and osteopenic/osteoporotic conditions are significant at all the regions. The only significant difference between pairs of regions occurs at the seventh decade between epicondyle and condyle.

The trabecular density (FIG. 12B) decreases with age within each specific site. The trabecular density decreases along the ranking of epicondyle, condyle and middle region between condyle and epicondyle during the sixth and seventh decade under normal and osteoporotic conditions. All differences between pairs of regions for a given decade and between decades for a given region are significant (p<0.01) except between epicondyle and condyle at the seventh decade under osteopenic/osteoporotic conditions.

### DISCUSSION

The rarefaction of the trabecular microstructure that occurs with age was investigated in the distal femur by measurement of trabecular thickness and trabecular density. These two parameters decrease with age at rates that differ from each other at any given site of the distal femur under normal BMD. The presence of osteopenia and osteoporosis increases such rates differently by site and by age.

The restriction of the trabecular analysis to the plane of the section arises from the focus of the study on the thickness of the trabecular rods and the incidence of trabeculae that lie parallel to the plane of the longitudinal section and appear on focus on the plane of the section. Because each longitudinal section was consistently cut through the center of the femoral head, along the femoral neck axis, the assumption of the study that allowed the comparison among femurs is that the trabecular orientation is consistent with the orientation of the cut. What justifies this assumption is the expectation that the biomechanical function of trabeculae in the distal femur be consistent with the orientation of the trabeculae by site (Koch, 1917; Tobin, 1955; Morgan et al., 2004; Patel and Murphy, 2006; Nazarian et al., 2007). Such restriction can be partially lifted by conducting a microCT scan on smaller specific sites of interest. The restriction of the analysis of the trabecular thickness to so-called rods is due to the rod and plate structure of the distal femur, and arises from the elimination of plates from this two-dimensional study because they may lie off the plane of the cut, thereby preventing accurate measurement (Parfitt et al., 1983; Odgaard, 1997; Gentzsch et al., 2003; Stauber and Muller, 2006a and 2006b). Because this is a two-dimensional study and usually plates are oriented off the plane through the femoral neck axis and through the center of the head, plates needed to be excluded from the assessment of the trabecular thickness. The method can be adapted to include three-dimensional data, although a two-dimensional approach is preferred for its relative simplicity and suitable results.

The microstructural differences observed among sites in terms of age are interpreted in terms of Pauwel's assessment of stresses per site by photoelastic experiments (Pauwels, 1965). Pauwel's model shows that the stresses are greatest at the medial and lateral sides and the smallest stresses are found at the central region. Moreover, the model shows that the trabecular distribution tends to follow the line of action of the force distribution acting on the distal femur. The mechanical function of trabeculae is more relevant at a high than at a low stress site. Roux in 1896 (Roux, 1896) was the first to hypothesize that the trabecular thickness and the stress magnitude at the site should be correlated.

The central area between the epicondyles, which biomechanically corresponds to the neutral axis, shows a lower trabecular thickness and trabecular density than the epicondyles which do not differ significantly between them in terms of trabecular thickness but do differ in terms of trabecular density during the sixth decade. As age increases, the bone density decreases in each of the three areas because of trabecular thinning. Mean trabecular thickness of each area within the distal femur is expected to be smaller than the mean trabecular thickness of all proximal femur sites because distal trabeculae are exclusively mechanically loaded along the femoral axis. The higher thickness of proximal trabeculae is explained by the bending stress that proximal trabeculae need to withstand.

The above results indicate that in the distal femur's sixth decade of life, trabecular density and trabecular thickness are highest at the sites of maximum stress, where the trabeculae are therefore hypothesized to have a function more mechanical than metabolic. Trabeculae, which are less exposed to mechanical stresses and hence perhaps with more of a metabolic function, may disappear in earlier decades in normal bone than in osteoporotic bone. In addition, reduction of bone density and reduction of trabecular number which in general may or may not be associated, shows that here the two phenomena are correlated.

Concerning post-menopausal bone, the higher fracture risk present in osteoporotic bone relative to that of normal bone (Burr et al., 1997) leads to the hypothesis that the decline in bone density and of trabecular thickness, which was observed in this study within the sixth and seventh decades, and which exert a mechanical function, takes place in earlier decades at the higher rates in osteoporotic rather than normal bone. This would explain the frequency of occurrence of fractures.

All references cited and/or discussed in this specification are incorporated herein by reference in their entireties and to the same extent as if each reference was individually incorporated by reference.

Abrahamsen B, Vestergaard P, Rud B, Bärenholdt O, Jensen J E, Nielsen S P, Mosekilde L, Brixen K 2006 Ten-year absolute risk of osteoporotic fractures according to BMD T score at menopause: the Danish Osteoporosis Prevention Study. J Bone Miner Res 21:796-800.

Adam C, Eckstein F, Milz S, Putz R 1998 The distribution of cartilage thickness within the joints of the lower limb of elderly individuals. J Anat 193:203-14.

Akizuki S, Mow V C, Muller F, Pita J C, Howell D S, Manicourt D H 1986 Tensile Properties of Human Knee Joint Cartilage: Influence of Ionic Conditions, Weight Bearing, and Fibrillation on the Tensile Modulus. J Orthopaedic Res 4:379-92.

Ascenzi, A. (1988) The micromechanics versus the macromechanics of cortical bone—A comprehensive presentation. J. of Biomech. Eng., 110, 357.

Ascenzi, M.-G., Ascenzi, A., Burghammer, M., Panzavolta, S., Benvenuti, A. and Bigi, A. (2003) Structural differences between “dark” and “bright” isolated human osteonic lamellae. Journal of Structural Biology, 141, 22-33.

Ascenzi, M.-G., Benvenuti, A., and Ascenzi, A. (2000) Single osteon micromechanical testing. In: Mechanical testing of bone, Y. An and R. Draughn, Eds, CRC Press, Boca Raton, Fla., 271.

Ascenzi, M.-G., Bahan, D. T. and Kelly, T. L. (1995) Precision in fan beam densitometrty: a multi-site validation. Hologic internal documentation.

Bauer J S, Kohlmann S, Eckstein F, Mueller D, Lochmuller E M, Link T M 2006 Structural analysis of trabecular bone of the proximal femur using multislice computer tomography: a comparison with dual X-ray absorptiometry for predicting biomechanical strength in vitro. Calc Tissue Int 78:78-89.

Beck T J, Looker A C, Mourtada F, Daphtary M M, Ruff C B 2006 Age trends in femur stresses from a simulated fall on the hip among men and women: evidence of homeostatic adaptation underlying the decline in hip BMD. J Bone Miner Res 21:1425-1432.

Bergmann, G., Deuretzbacher, G., Heller, M., Graicen, F., Rohlmann, A., Strauss, J., Duda, G. N., 2001. Hip contact forces and gait patterns from routine activities. J. Biomech. 34, 859-871.

Bini, F., Marinozzi, A., Marinozzi, F. and Patanè, F. (2002) Microtensile measurements of single trrabeculae stiffness in human femur. Calc. Tissue Int., 35, 1515.

Boden, B P, Speer K P (1997) Femoral stress fractures. Clin. Sports Med., 16: 307.

Boonen S, Aerssens J, Broos P, Pelemans W, Dequeker J 1995 Age-related bone loss and senile osteoporosis: evidence for both secondary hyperparathyroidism and skeletal growth factor deficiency in the elderly. Aging 7:414-422.

Bousson V, Le Bras A, Roqueplan F, Kang Y, Mitton D, Kolta S, Bergot C, Skalli W, Vicaut E, Kalender W, Engelke K, Laredo J D 2006 Volumetric quantitative computer tomography of the proximal femur: relationships linking geometric and densitometric variables to bone strength. Role for compact bone. Osteoporos Int 17: 855-864.

Boyde A 1984 Methodology of calcified tissue specimen preparation for scanning electron microscopy. In: Dickson G R (ed.) Methods of Calcified Tissue Preparation. Elsevier Science Publishers, New York, N.Y., USA, pp. 251-307.

Brenneman S K, Barrett-Connor E, Sajjan S, Markson L E, Siris E S 2006 Impact of recent fracture on health-related quality of life in postmenopausal women. J Bone Miner Res 21:809-816.

Burr, D B, Forwood, M R, Fyhrie, D P, Martin, R B, Shaffler, M B and Turner, C H (1997) Bone microdamage and skeletal fragility in osteoporotic and stress fractures. J. Bone Min. Res., 12, 6.

Cann C. E. (1988) Quantitative CT for determination of bone mineral density; a review. Radiology, 166, 509.

Chan A H W, Crowder C M, Rogers T L 2007 Variation in cortical bone histology within the human femur and its impact on estimating age at death. Amer J Phys Anthrop 132:80-8.

Chappard C, Brunet-Imbault B, Lemineur G, Giraudeau B, Basillais A, Harba R, Benhamou C L, 2005. Anisotropy changes in post-menopausal osteoporosis: characterization by a new index applied to trabecular bone radiographic images. Osteoporos Int 16:1193-1202.

Cheng, X. G., Lowet, G., Boonen, S., Nicholson, P. H. F., Brys, P., Nijs, J., Dequeker, J., 1997. Assessment of the strength of proximal femur in vitro: relationship to femoral bone mineral density and femoral geometry. Bone 20, 213-218.

Cummings S. R., Black, D., Nevitt, M., Browner, M., Cauley, J., Ensrud, K., Genant, H. K., Palermo, L., Scott, J. and Vogt, T. (1993) Bone density at various sites for prediction of hip fractures. The study of Osteoporotic Fractures Research Group. Lancet, 341, 72.

Cummings, S R, Nevitt, N, Browner, W (1995) Risk factors for hip fracture in white women. Study of Osteoporotic Fractures Research Group. New Engl. J. Med., 332, 767.

Cummings S R, Cawthon P M, Ensrud K E, Cauley J A, Fink H A, Orwoll E S 2006 BMD and risk of hip and nonvertebral fractures in older men: a prospective study and comparison with older women. J Bone Miner Res 21:1550-1556.

Dequeker, J (1994), Assessment of quality of bone in osteoporosis-Biomed I: Fundamental study of relevant bone. Clin Rheum 13:8-12.

Fogelman I, Rodin A, Blake G 1989 Impact of bone mineral measurements on osteoporosis. Eur J Nucl Med 16:39-52.

Garden, R. S. (1961) The structure and function of the proximal end of the femur. J. Bone and Joint Surg., 43-B, 576.

Gentzsch C, Delling G, Kaiser E 2003 Microstructural classification of resoprtion lacunae and perforations in human proximal femora. Calcif Tissue Int 72:698-709.

Giangregorio L, Blimkie C J (2002) Skeletal adaptations to alterations in weight-bearing activity: a comparison of models of disuse osteoporosis. Sports Med., 32:459.

Glorioso J E, Ross G, Leadbetter W B (2002) Femoral supracondular stress fractures—An unusual cause of knee pain, The Physiology and Sports Medicine 30, 9 on line publication.

Havill, L M, Mahaney, M C, L Binkley, T, Specker B L 2007. Effects of genes, sex, age, and activity on BMC, bone size, and areal and volumetric BMD. J Bone Min Res 22:737-746.

Heller, M. O., Bergmann, G., Deuretzbacher, G., Durselen, L., Pohl, M., Claes, L., Haas, N. P., Duda, G. N., 2001. Musculo-skeletal loading conditions at the hip during walking and stair climbing. J. Biomech. 34, 883-893.

Hildebrand T, Laib A, Müller R, Dequeker J, Ruegsegger P 1999 Direct three-dimensional morphometric analysis of human cancellous bone: Microstructural data from spine, femur, iliac crest, and calcaneus. J Bone Miner Res 14:1167-1174.

Ho, C. P., Kim, R. W., Schaffler, M. B. and Sartoris, D. J. (1990) Accuracy of dual-energy radiographic absorptiometry of the lumbar spine:cadaver study. Radiology, 176, 171.

Hologic Inc. 1996 Hologic QDR-4500 user's guide. Hologic Inc., Waltham, Mass.

Hui, S., Slemenda, C. W. and Johnston, C. C. (1988) Age and bone mass as predictor of fracture in a prospective study. J. Clin. Invest., 81, 1804-1809.

Hui, S. L., Slemenda, C. W., Johnston, C. C. Jr., 1988. Age and bone mass as predictors of fracture in a prospective study. J. Clin. Invest. 81, 1804-1809.

Issever, A. S., Vieth, V., Lotter, A., Meier, N., Laib, A., Newitt, D., Majumdar, S., Link, T. M., 2002. Local differences in the trabecular bone structure of the proximal femur depicted with high-spatial resolution MR imaging and multisection CT. Acad Radiol. 9, 1395-1406.

Jenson F, Padilla F, Bousson V, Bergot C, Laredo J D, Laugier P 2006 In vitro ultrasonic characterization of human cancellous femoral bone using transmission and backscatter measurements: relationships to bone mineral density. J Acoust Soc Am 119:654-663.

Kaptoge S, Jakes R W, Dalzell N, Wareham N, Khaw K T, Loveridge N, Beck T J, Reeve J 2007 Effects of physical activity on evolution of proximal femur structure in a younger elderly population. Bone 40:506-515.

Khosla S, Riggs B L, Atkinson E J, Oberg A L, McDaniel L J, Holets M, Peterson J M, Melton III, L J 2006 Effects of sex and age on bone microstructure at the ultradistal radius: A population-based noninvasive in vivo assessment. J Bone Miner Res 21:1-8.

Kingsmill, V. J. and Boyde, A. (1998) Mineralization density of human mandibular bone: quantitative backscattered electron image analysis. J. Anat., 192, 245.

Koch J 1917 The laws of bone architecture. Am J Anat 21:177-298.

Kothari, M., Keaveny, T. M., Lin, J. C., Newitt, D. C., Genant, H. K., Majumdar, S., 1998. Impact of spatial resolution on the prediction of trabecular architecture parameters. Bone 22, 437-443.

Krieg M A, Comuz J, Ruffieux C, Van Melle G, Büche D, Dambacher M A, Hans D, Hartl F, Häuselmann H J, Kraenzlin M, Lippuner K, Neff M, Pancaldi P, Rizzoli R, Tanzi F, Theiler R, Tyndall A, Wimpfheimer C, Burckhardt P 2006 Prediction of hip fracture risk by quantitative ultrasound in more than 7000 Swiss women > or =70 years of age: comparison of three technologically different bone ultrasound devices in the SEMOF study. J Bone Miner Res 21:1457-1463.

Krug, R., Banerjee, S., Han, E. T., Newitt, D. C., Link, T. M., Majumdar, S., 2005. Feasibility of in vivo structureal analysis of high-resolution magnetic resonance images of the proximal femur. Osteoporos Int., 16, 1307-1314.

Kuiper, J. W., van Kujik, C., Grashuis, J. L., Ederveen, A. G. H. and Schütte (1996) Accuracy and the influence of marrow fat on quantitative CT and dual-energy X-ray absorptiometry measurements of the femoral neck in vitro. Osteoporosis Int., 6, 25.

Leichter I, Bivas A, Giveon A, Margulies J Y, Weinreb A 1987 The relative significance of trabecular and cortical bone density as a diagnostic index for osteoporosis. Phys Med Biol 32:1167-1174.

Lieberman U A, Weiss S R, Bröll J, Minne H W, Quan H, Bell N H, Rodriguez-Portales J, Downs R W, Dequeker J, Favus M 1995. Effect of oral alendronate on bone mineral density and the incidence of fractures in postmenopausal osteoporosis. The Alendronate Phase III Osteoporosis Treatment Study Group. New Eng J Med 333:1437-1443.

Lilley J, Walters B G, Heath D A, Drolc Z 1991 In vivo and in vitro precision for bone density measured by dual-energy x-ray absorption. Osteop Int 1: 141-146.

Link, T. M., Majumdar, S., Grampp, S., Guglielmi, G., van Kuijk, C., Imhof, I., Glueer, C. and Adams, J. (1999) Imaging of trabecular bone structure in osteoporosis. Eur. Radiol., 9, 1781.

Link, T M, Vieth, V, Langenberg, R, Mayer, N, Lotter, A, Newitt, D and Majumdar, S (2002) Structure analysis of high resolution magnetic resonance imaging of proximal femur: in vitro correlation with biomechanical strength and BMD. Calc. Tissue Int., Online publication, October 10.

Link T M, Vieth V, Langenberg R, Meier N, Lotter A, Newitt D, Majumdar S, 2003. Structure analysis of high resolution magnetic resonance imaging of the proximal femur: in vitro correlation with biomechanical strength and BMD. Calcif Tissue Int. 72, 156-165.

Looker A C, Wahner H W, Dunn W L, Calvo M S, Harris T B, Hayse S P, Johnston Jr C C, Lindsay R L 1995 Proximal femur bone mineral levels of US adults. Osteop Int 5:389-409.

Lozupone, E. and Favia A. (1982) Density of trabecular framework and osteogenic activity in the spongiosa of long bones subjected to drastic changes in mechanical loading in the dog. Anat. Anz. Jena, 152, 245.

Lozupone, E. and Favia A. (1988) Distribution of resorption process in the compacta and spongiosa of bones from lactating rats fed a low-calciun diet. Bone, 9, 215.

Lozupone, E. and Favia A. (1990) The structure of the trabeculae of cancellous bone. II. Long bones and mastoid. Calc. Tissue Int., 46, 367.

Lundeen G A, Knecht S L, Vajda E G, Bloebaum R D, Hofmann AbA 2001 The contribution of cortical and cancellous bone to dual-energy x-ray absorptiometry measurements in the female proximal femur. Osteop Int 12:192-198.

Madore G R 2006 Fractures, Hips. eMedicine from Web M D [serial online], available at http://www.emedicine.com/emerg/topic198.htm. Accessed May 31, 2006.

Marín F, González-Macías J, Díez-Pérez A, Palma S, Delgado-Rodríguez M 2006 Relationship between bone quantitative ultrasound and fractures: a meta-analysis. J Bone Miner Res 21:1126-1135.

Marshall L M, Lang T F, Lambert L C, Zmuda J M, Ensrud K E, Orwell E S 2006 Dimensions and volumetric BMD of the proximal femur and their relation to age among older U.S. men. J Bone Min Res 21:1197-1206.

Matheson G O, Clement D B, McKenzie D C (1987): Stress fractures in athletes: a study of 320 cases. Am. J. Sports Med., 15: 46.

Mazess R, Chesnut III C H, McClung M, Genant H 1992 Enhanced precision with dual-energy x-ray absorptiometry. Calcif Tissue Int 51:14-17.

Meta M, Lu Y, Keyak J H, Lang T 2006 Young-elderly differences in bone density, geometry and strength indices depend on proximal femur sub-region: a cross sectional study in Caucasian-American women. Bone 39:152-158.

Morgan, E. F., Bayraktar, H. H., Yeh, O. C., Majumdar, S., Burghardt, A., Keaveny, T. M., 2004. Contribution of inter-site variations in achitechture to trabecular bone apparent yield strains. J Biomech. 37, 1413-1420.

Nazarian A, Müller J, Zurakowski D, Müller R, Snyder B D 2007 Densitometric, morphometric and mechanical distributions in the human proximal femur. J Biomech. Published online Jan. 25, 2007; doi: 10.1016/j.jbiomech.2006.11.022.

Nurzenski M K, Briffa N K, Price R I, Khoo B C, Devine A, Beck T J, Prince R L 2007. Geometric indices of bone strength are associated with physical activity and dietary calcium intake in healthy older women. J Bone Min Res 22: 416-424.

Odgaard A 1997 Three-dimensional methods for quantification of cancellous bone architecture. Bone 20:315-328.

Odgaard, A. (2001) Quantification of cancellous bone architecture. In: Bone Mechanics Handbook, S. C. Cowin, Ed., 14-1, CRC Press, Boca Raton, Fla.

Parfitt, A. M. (1983) Stereologic basis of bone histomorphometry; theory of quantitative microscopic and reconstruction of the third dimension. In Bone histomorphometry: techniques and interpretation. Robert R. Recker, Ed. CRC Press, Inc. Boca Raton, Fla., 53-87, 92.

Parfitt A M, Mathews C H E, Villanueva A R, Kleerekoper M, Frame B, Rao D S 1983 Relationships between surface, volume, and thickness of iliac trabecular bone in aging and in osteoporosis: Implications for the microanatomic and cellular mechanisms of bone loss. J. Clin Invest 72:1396-1409.

Patel V, Issever A S, Burghardt A, Laib A, Ries M, Majumdar S 2003 MicroCT evaluation of normal and osteoarthritic bone structure in human knee specimens. J Orthopaedic Res 21:6-13.

Patel S, Murphy K P 2006 Fractures of the proximal femur: correlates of radiological evidence of osteoporosis. Skeletal Radiol 35:202-211.

Pauwels, F. (1965) Gesammelte Abhandlungen zur funktionellen Anatomie des Bewegungsapparates. Springer-Verlag, Berlin.

Pawels, F. (1965) Über die Verteilung der Spongiosadichte im coaxalen Femurende und ihre Bedeutung fur die Lehre vom funktionellen Bau des Knochens. Siebenter Beitrag zur funktionellen Anatomie und kausalen Morphologie des Stützapparates. Gegenbaurs morphol. Jahrb., 95, 35.

Pulkkinen P, Eckstein F, Lochmüller E M, Kuhn V, Jämsä T 2006 Association of geometric factors and failure load level with the distribution of cervical vs. trochanteric hip fractures. J Bone Miner Res 21:895-901.

Reddy, G. K., Stehno-Bittel, L., Hamade, S., Enwemeka, C. S., 2001. The biomechanical integrity of bone in experimental diabetes. Diabetes Res Clin Pract. 54, 1-8.

Riggs, B., Hodgson, W. and O'Fallon, M. (1990) Effect of fluoride treatment on the fracture rate in postmenopausal women with osteoporosis. New Engl. J. Med., 322, 802.

Ross, P., Davis, J., Wasnich, R. and Vogel, J. (1990) A critical review of bone mass and the risk of fractures in osteoporosis. Calcif. Tissue Int., 46, 149.

Roux, W. (1895) Gesammelte Abh. 1. W. Engelmann, 700.

Scolamacchia, B. (1999) Rarefazione della spugnosa del femore in rapporto all'età. Tesi di laurea in Scienze Biologiche, Università di Bari, Angela Favia thesis advisor.

Seeman E, Delmas P D. 2006. Bone quality—the material and structural basis of bone strength and fragility. N Eng J Med 354: 2250-2261.

Sell, C. A., Masi, J. N., Burghardt, A., Newitt, D., Link, T. M., Majumdar, S., 2005. Quantification of trabecular bone structure using magnetic resonance imaging at 3 tesla-calibration studies using microcomputed tomography as a standard reference. Calicif Tissue Int. 76, 355-364.

Singh M, Nagrath A R, Maini P S 1970 Changes in trabecular pattern of the upper end of the femur as an index of osteoporosis. J Bone Joint Surg 52-A:457-467.

Sornay-Rendu E, Boutroy S, Munoz F, Delmas P D 2007 Alterations of cortical and trabecular architecture are associated with fractures in postmenopausal women, partially independent of decreased BMD measured by DEXA: the OFELY study. J Bone Min Res 22:425-33.

Stauber, M., Muller, R., 2006. Age-related changes in trabecular bone microstructures: global and local morphometry. Osteoporos Int. 17, 616-626.

Stauber M, Müller R 2006 Volumetric spatial decomposition of trabecular bone into rods and plates—A new method for local bone morphometry. Bone 38:475-484.

Stenstrom M., Olander, B., Lehto-Axtelius, D., Madsen, J. and Nordsletten, L. and Carlsson, G. (2000) Bone mineral density and bone structure parameters as predictors of bone strength: an analysis using computerized microtomography and gastrectomy-induced osteopenia in the rat. J. Biomech., 33, 289.

Tissakht M, Ahmed A M, Chan K C 1996 Calculated Stress-Shielding in the Distal Femur after Total Knee Replacement Corresponds to the Reported Location of Bone Loss. J Orthopaedic Res 14:778-85.

Tobin, W. J. (1955) The internal architecture of the femur and its clinical relevance. J. Bone & Joint Surg., 37 A, 57-72.

Tothill P 1989 Methods of bone mineral measurement. Phys Med Biol 34:543-572.

Tsangari H, Findlay D M, Zannettino A C W, Pan B, Kuliwaba J S, Fazzalari N L 2006 Evidence for reduced bone formation surface relative to bone resorption surface in female femoral fragility fracture patients. Bone 39:1226-1235.

Tsangari H, Findlay D M, Fazzalari N L 2007 Structural and remodeling indices in the cancellous bone of the proximal femur across adulthood. Bone 40:211-217.

Ulrich D, van Rietberen B, Laib A, Ruegsegger P, 1999. The ability of three-dimensional structural indices to reflect mechanical aspects of trabecular bone. Bone 25, 55-60.

Van Lenthe G H, De Waal Malefut M C, Huiskes R 1997 Stress shielding after total knee replacement may cause bone resorption in the distal femur. J Bone Joint Surg 79-B:117-22.

Van Rietbergen, B., Huiskes, R., Eckstein, F., Ruesegger, P., 2003. Trabecular bone tissue strains in the healthy and osteoporotic human femur. J Bone Miner Res. 18, 1781-1788.

Ward, F. 0. (1838) Outline of Human Osteology. Henry Renshaw, London.

Wasnich R D 1996 Vertebral fracture epidemiology. Bone 18, 79S-183S.

Weiss L A, Barrett-Connor E, von Mühlen D, Clark P 2006 Leptin predicts BMD and bone resorption in older women but not older men: the Rancho Bernardo study. J Bone Miner Res 21:758-764.

Whitehouse, W. J. and Dyson, D. (1974) Scanning electron microscope studies of trabecular bone in the proximal end of the human femur. J. Anat., 118, 417.

Zuckerman J D 1996 Hip fracture. New Eng J. Med 334:1519-1525.

All references cited herein are incorporated by reference in their entireties as though each were incorporated by reference individually.

1. A method of creating a bone evaluation template, the method comprising:
(a) identifying one or more regions of a subject bone;
(b) obtaining at least one control bone corresponding to the subject bone and having at least one selected region corresponding to a selected region of the subject bone;
(c) obtaining a digitally scanned image of a trabecular structure of each selected region of each control bone;
(d) identifying one or more trabecular families within each selected region of each control bone;
(e) identifying one or more microstructural sites based on the trabecular families;
(f) determining a percent trabecular density and an average trabecular thickness in each of the microstructural sites;
(g) subdividing each selected region of each control bone into sections of related trabecular structure based on the trabecular families, trabecular densities, and trabecular thicknesses; and
(h) based on the sections of related trabecular structure, creating a bone evaluation template for evaluating the subject bone.

2. The method of claim 1, wherein the subject bone and the at least one control bone are in the same decade of age.

3. The method of claim 1, wherein the subject bone and the at least one control bone are of the same gender.

4. The method of claim 1, wherein the subject bone and the at least one control bone show signs of osteoporosis.

5. The method of claim 1, wherein the control bone is a proximal femur.

6. The method of claim 5, wherein at least one of the microstructural sites is selected from central head, epiphyseal head, femoral neck, greater trochanter, intermediate region, and Ward's triangle.

7. The method of claim 5, wherein at least one trabecular family is selected from the right, bent, medial, and greater trochanter family.

8. The method of claim 1, wherein the control bone is a distal femur.

9. The method of claim 8, wherein at least one of the microstructural sites is selected from epicondyle, condyle, and middle region.

10. The method of claim 1, wherein the digitally scanned image of the control bone is obtained using a regular light microscopy technique capable of producing high resolution digital images.

11. The method of claim 1, wherein the digitally scanned image of the subject bone is obtained using a clinical imaging technique selected from DEXA, CT, MicroCT, MRI, and ultrasound.

12. The method of claim 1, wherein the values of average trabecular density and percent trabecular thickness in the related trabecular structure are each independently relatively homogeneous.

13. The method of claim 1, wherein trabecular thickness and trabecular density are assessed using semi-automatic histomorphometry.

14. The method of claim 1, comprising the steps of:
displaying the subdivided sections to form a template; and
superimposing the template onto a digitally scanned image of the subject bone.

15. A bone evaluation template, comprising:
a shape representative of at least one selected region of at least one control bone corresponding to a selected region of a subject bone; and
a plurality of subsections displayed within the shape that are representative of empirical data obtained from observing at least one digitally scanned image of the at least one selected region of the at least one control bone;
wherein the empirical data corresponds to values of average trabecular thickness and percent trabecular density in one or more trabecular families within each selected region of each control bone and in one or more microstructural sites within each selected region of each control bone, and
wherein, when the template is superimposed onto a digitally scanned image of the selected region of the subject bone, the values of average trabecular thickness and percent trabecular density of the selected region of subject bone are independently relatively homogeneous within each subsection.

16. The bone evaluation template of claim 15, wherein the subject bone and the at least one control bone are in the same decade of age.

17. The bone evaluation template of claim 15, wherein the subject bone and the at least one control bone are of the same gender.

18. The bone evaluation template of claim 15, wherein the subject bone and the at least one control bone show signs of osteoporosis.

19. The bone evaluation template of claim 15, wherein the digitally scanned image of the control bone is obtained using a regular light microscopy technique capable of producing high resolution digital images.

20. The bone evaluation template of claim 15, wherein the digitally scanned image of the subject bone is obtained using a clinical imaging technique selected from DEXA, CT, MicroCT, MRI, and ultrasound.

21. A method of evaluating bone quality, comprising:
(a) identifying one or more regions of a subject bone;
(b) obtaining a bone evaluation template comprising
(1) a shape representative of at least one selected region of at least one control bone corresponding to a selected region of a subject bone; and
(2) a plurality of subsections displayed within the shape that are representative of empirical data obtained from observing at least one digitally scanned image of at least one selected region of the at least one control bone, wherein the empirical data corresponds to values of average trabecular thickness and percent trabecular density in one or more trabecular families within each selected region of each control bone and in one or more microstructural sites within each selected region of each control bone,
(c) obtaining a digitally scanned image of a selected region of the subject bone;
(d) superimposing the template onto the digitally scanned image of the subject bone, wherein values of average trabecular thickness and percent trabecular density of the selected region of the subject bone are independently relatively homogeneous within each subsection; and
(e) comparing values of average trabecular thickness and percent trabecular density within each subsection to evaluate bone quality in terms of relative strengths of the trabecular microstructure of the selected region of the subject bone.

22. The method of claim 21, wherein the subject bone and the at least one control bone are in the same decade of age.

23. The method of claim 21, wherein the subject bone and the at least one control bone are of the same gender.

24. The method of claim 21, wherein the subject bone and the at least one control bone show signs of osteoporosis.

25. The method of claim 21, wherein the digitally scanned image of the control bone is obtained using a regular light microscopy technique capable of producing high resolution digital images.

26. The method of claim 21, wherein the digitally scanned image of the subject bone is obtained using a clinical imaging technique selected from DEXA, CT, MicroCT, MRI, and ultrasound.

27. The method of claim 1, wherein the control bone is a cadaveric bone.

28. The method of claim 15, wherein the control bone is a cadaveric bone.

29. The method of claim 21, wherein the control bone is a cadaveric bone.
